(19) United States
(12) Patent Application Publication
(10) Pub. No.: US 2007/0207144 A1
(43) Pub. Date: Sep. 6, 2007

(54) Ubiquitin/proteasome inhibitors for the treatment of spinal muscular atrophy

(76) Inventor: Lee Rubin, Wellesley, MA (US)

Correspondence Address: COOLEY GODWARD KRONISH LLP;ATTN: Patent Group, Suite 500, 1200 - 19th Street, NW, WASHINGTON, DC, 20036-2402, US

(21) Appl. No.: 11/706,253

(22) Filed: Feb. 15, 2007

Related U.S. Application Data
(60) Provisional application No. 60/774,015, filed on Feb. 16, 2006.

(51) Int. Cl. A61K 39/395; A61K 38/16; A61K 31/69

(52) U.S. Cl. 424/133.1; 514/2; 514/64; 514/18; 514/7; 514/412; 514/424

(57) ABSTRACT

The present invention provides compositions and method for the treatment of spinal muscular atrophy comprising administering a therapeutically effective amount of a therapeutically amount of at least one proteasome inhibitor to a subject in need of treatment of spinal muscular atrophy.

### BACKGROUND OF THE INVENTION

Proximal spinal muscular atrophy (SMA) is a clinically heterogeneous group of neuromuscular disorders characterized by degeneration of the anterior horn cells of the spinal cord. Patients suffer from symmetrical weakness of trunk and limb muscles, the legs being more affected than the arms and the proximal muscles weaker than the distal ones; diaphragm, facial and ocular muscles are spared. There are three forms of childhood-onset SMA (types I, II and III) can be distinguished on the basis of age of onset and severity of the clinical course assessed by clinical examination, muscle biopsy and electromyography (EMG) (Munsat T L, Davies K E (1992).

Type I (Werdnig-Hoffmann disease) is the most acute and severe form, with onset before six months and death usually before two years; children are never able to sit without support. Symptoms of the disease can be present in utero, as reduction of fetal movements, at birth, or appear more often within the first four months of life. Children affected are particularly floppy with feeding difficulties and diaphragmatic breathing. Death is generally due to respiratory insufficiency.

Type II (intermediate, chronic form) has onset between six and eighteen months of age; muscular fasciculations are common, and tendon reflexes progressively reduce. Children are unable to stand or walk without aid. Most of patients generally develop a progressive muscular scoliosis which can require surgical correction through

Type III (Kugelberg-Welander disease) is a mild, chronic form, with onset after the age of 18 months; motor milestones achievement is normal, and deambulation can be preserved until variable ages. Life expectancy is almost normal but quality of life is markedly compromised.

From a genetic point of view, SMA is an autosomal recessive condition, caused by disruption of SMN1 gene, located in 5q13 (Lefebvre S., Burglen L., Reboullet S., Clermont O., Burlet P., Viollet L., Benichou B., Cruaud C., Millasseau P., Zeviani M., Le Paslier D., Frezal J., Cohen D., Weissenbach J., Munnich A., Melki J. (1995). Cell 80: 155-165). This gene is absent in the majority of patients (95%), and small intragenic mutations have been described in 2-3% of cases. The incidence of the disease varies from 1/6000 to 1/10000, being healthy carriers quite common ( 1/40- 1/50) in general population (Wirth B., Schmidt T., Hahnen E., Rudnik-Schoneborn S., Krawczak M., Muller-Myhsok B., Schonling J., Zerres K. (1997). Am. J. Hum. Genet., 61: 1102-1111.).

At the genomic level, only five nucleotides have been found that differentiate the SMN1 gene from the SMN2 gene. Furthermore, the two genes produce identical mRNAs, except for a silent nucleotide change in exon 7, namely, a C→T change six base pairs inside exon 7 in SMN2 as compared to SMN1. This mutation modulates the activity of an exon splicing enhancer (Lorson and Androphy (2000) Hum. Mol. Genet. 9:259-265). The result of this and the other nucleotide changes in the intronic and promoter regions is that most SMN2 transcripts lack exons 3, 5, or 7. In contrast, the mRNA transcribed from the SMN1 gene is generally a full-length mRNA with only a small fraction of its transcripts spliced to remove exon 3, 5, or 7 (Gennarelli et al. (1995) Biochem. Biophys. Res. Commun. 213:342-348; Jong et al. (2000) J. Neurol. Sci. 173:147-153). All patients have at least one, generally two to four copies of the SMN2 gene which is nearly identical to SMN1, and encodes the same protein. However, the SMN2 gene produce only low levels of full-length SMN protein. The clinical severity of SMA patients inversely correlates with the number of SMN2 genes and with the level of functional SMN protein produced (Lorson C L, Hahnen E, Androphy E J, Wirth B. Proc Natl Acad Sci 1999; 96:6307-6311. Vitali T. Sossi V, Tiziano F, et al. Hum Mol Genet 1999; 8:2525-2532. Brahe C. Neuromusc. Disord. 2000; 10:274-275. Feldkotter M, Schwarzer V, Wirth R, Wienker T I, Wirth B. Am J Hum Genet 2002; 70:358-368. Lefebvre S, Burlet P, Liu Q, et al. Nature Genet 1997; 16:265-269. Coovert D D, Le T T, McAndrew P E, et al. Hum Mol Genet 1997; 6:1205-1214. Patrizi A L, Tiziano F, Zappata S, Donati A, Neri G, Brahe C. Eur J Hum Genet 1999; 7:301-309.)

In the course of studies of the functions of heterogeneous nuclear ribonucleoproteins (hnRNPs) (Dreyfuss et al., 1993, Ann. Rev. Biochem. 62:289-321), it was found that the SMN protein interacts with fibrillarin, an RNA-binding protein involved in rRNA processing, and with several other RNA-binding proteins (Liu and Dreyfuss, 1996, EMBO J. 15:3555-3565). Monoclonal antibodies to SMN localized the protein to a unique cellular location. SMN exhibits a general localization in the cytoplasm and is particularly concentrated in several prominent nuclear bodies called gems (for gemini of coiled bodies). Gems are novel nuclear structures which are related in number and size to coiled bodies and are usually found in close proximity to them (Liu and Dreyfuss, 1996, EMBO J. 15:3555-3565). Coiled bodies, which were first described by Ramn y Cajal (1903, Trab. Lab. Invest. Biol. 2:129-221), are prominent nuclear bodies found in widely divergent organisms, including plant and animal cells (Bohmann et al., 1995, J. Cell Sci. 19:107-113; Gall et al., 1995, Dev. Genet. 16:25-35). Coiled bodies contain the spliceosomal U1, U2, U4/U6, and U5 snRNPs, U3 snoRNAs, and several proteins, including the specific marker p80-coilin, fibrillarin, and NOP140 (Bohmann et al., 1995, J. Cell Sci. 19:107-113, and references therein; Gall et al., 1995, Dev. Genet. 16:25-35). Expression of p80-coilin mutants and microscopic observations suggest a close association between coiled bodies and the nucleolus (Raska et al., 1990, J. Struct. Biol. 104:120-127; Andrade et al., 1991, J. Exp. Med. 173:1407-1419; Bohmann et al., 1995, J. Cell Biol. 131:817-831). However, the specific functions of coiled bodies are not clear. Current ideas propose that coiled bodies may be involved in processing, sorting, and assembly of snRNAs and snoRNAs in the nucleus. The close association of gems and coiled bodies raises the possibility that the SMN protein and gems are also involved in the processing and metabolism of small nuclear RNAs (Liu and Dreyfuss, 1996, EMBO J. 15:3555-3565).

The mechanism leading to motorneuron loss and to muscular atrophy still remains obscure, although the availability of animal models of the disease is rapidly increasing knowledge in this field (Frugier T, Tiziano F D, Cifuentes-Diaz C, Miniou P, Roblot N, Dierich A, Le Meur M, Melki J. (2000) Hum Mol. Genet. 9:849-58; Monani U R, Sendtner M, Coovert D D, Parsons D W, Andreassi C, Le T T, Jablonka S, Schrank B, Rossol W, Prior T W, Morris G E, Burghes A H. (2000) Hum Mol Genet 9:333-9; Hsieh-Li H M, Chang J G, Jong Y J, Wu M H, Wang N M, Tsai C H, Li H. (2000) Nat Genet 24:66-70; Jablonka S, Schrank B, Kralewski M, Rossbll W. Sendtner M. (2000) Hum Mol. Genet. 9:341-6). Also the function of SMN protein is still partially unknown, and studies indicate that it can be involved in mRNA metabolism (Meister G, Eggert C, Fischer U. (2002). Trends Cell Biol. 12:472-8; Pellizzoni L, Yong J, Dreyfuss G. (2002). Science. 298: 1775-9), and probably in transport of proteins/mRNA to neuromuscular junctions (Ci-fuentes-Diaz C, Nicole S, Velasco M E, Borra-Cebrian C, Panozzo C, Frugier T, Millet G, Roblot N, Joshi V, Melki J. (2002) Hum Mol. Genet. 11: 1439-47; Chan Y B, Miguel-Aliaga I, Franks C, Thomas N, Trulzsch B, Sattelle D B, Davies K E, van den Heuvel M. (2003) Hum Mol. Genet. 12:1367-76; McWhorter M L, Monani U R, Burghes A H, Beattie C E. (2003) J. Cell Biol. 162:919-31; Rossoll W, Jablonka S, Andreassi C, Kroning A K, Karle K, Monani U R, Sendtner M. (2003) J. Cell Biol. 163:801-812).

There is no cure for SMA available to date and therefore it is an object of the present invention to provide compositions and methods for the treatment of SMA.

### SUMMARY OF THE INVENTION

In one embodiment of the invention provides compounds, or pharmaceutically acceptable salt forms or prodrugs thereof, which are useful as inhibitors of ubiquitin/proteasome pathway for the manufacture of a medicament for the treatment of SMA.

It is another embodiment of the invention to provide pharmaceutically acceptable carrier and a therapeutically effective amount of at least one proteasome inhibitor, or pharmaceutically acceptable salt form or prodrug thereof for the manufacture of a medicament for the treatment of SMA.

It is another embodiment of the invention to provide a method for treating SMA comprising administering to a subject in need of such treatment a therapeutically effective amount of at least one compound described herein, in particular a proteasome inhibitor, or a pharmaceutically acceptable salt form or prodrug thereof.

The present invention is based on the discovery that proteasome inhibitors increase, not only the production of SMN protein, but additionally, the level of gems in fibroblasts isolated from an SMA patient.

Furthermore, the present invention relates to the use of the assays and screening methods described herein to identify SMA therapies. For example, compounds suspected to be proteasome inhibitors can be screened for activity in fibroblast cells for gem formation. In alternative examples, compounds that are suspected of modulating the gene expression of SMN exon 7 can be screened. The invention includes compounds identified by this screening technique and their methods of using the compounds to treat SMA.

### BRIEF DESCRIPTION OF THE DRAWINGS

FIG. 1 shows pictures of SMN & Gems induced by Velcade and Lactacystin as compared with the control DMSO in patient fibroblasts.

FIG. 2 shows pictures of SMN & Gems induced by different concentrations of Antiprotealide as compared with the control DMSO in patient fibroblasts.

FIG. 3 is a graph of percentage of cells with Gems vs. the different concentrations for peptide boronate proteasome inhibitors as compared with Lactacystin: MG-262 and PS-341 (Velcade®).

FIG. 4 is a graph of percentage of cells with Gems vs. the different concentrations for four different lactone proteasome inhibitors: Antiprotealide, Omuralide, α-Methyl clasto-Lactacystin β-Lactone, Lactacystin.

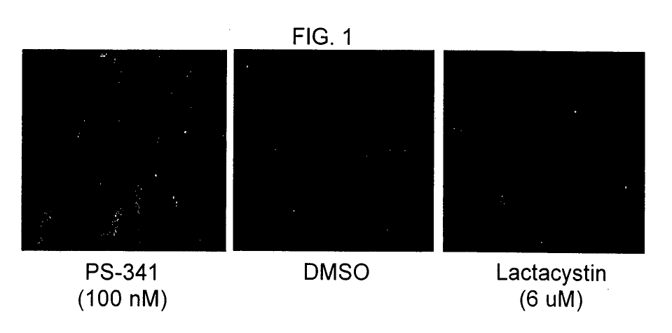

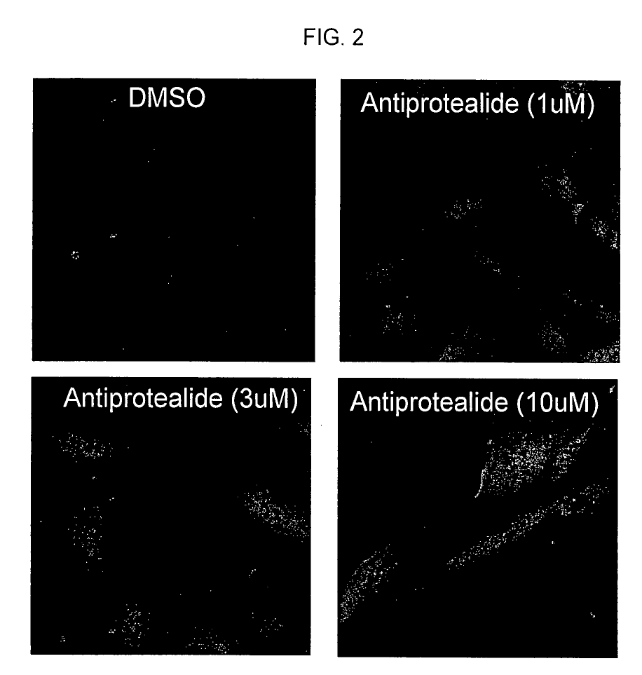

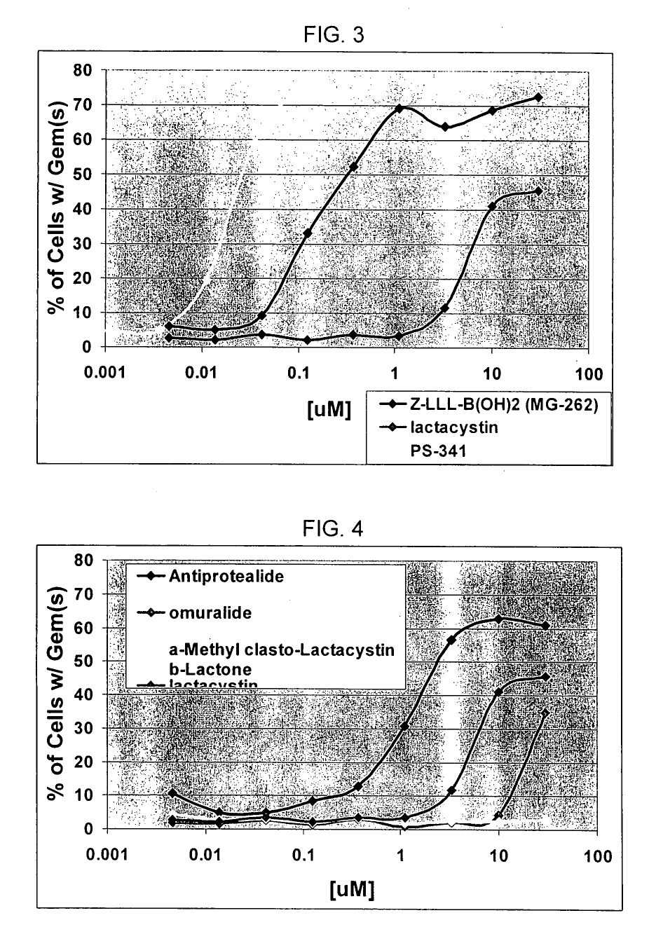

### DETAILED DESCRIPTION OF THE INVENTION

The first embodiment of the present invention is the composition and method for the treatment of spinal muscular atrophy (SMA) comprising at least one proteasome inhibitor or a pharmaceutically acceptable salt, isomer, prodrug, analog, metabolite or derivative thereof.

The second embodiment is the composition and method for the treatment of spinal muscular atrophy (SMA) comprising at least one proteasome inhibitor or a pharmaceutically acceptable salt, isomer, prodrug, analog, metabolite or derivative thereof and wherein the level of gemini of coiled bodies (gems) are increased in the SMA patient fibroblasts.

The proteasome, (also referred to as multicatalytic protease (MCP), multicatalytic proteinase, multicatalytic proteinase complex, multicatalytic endopeptidase complex, 20S, 26S, or ingensin) is a large, multiprotein complex present in both the cytoplasm and the nucleus of all eukaryotic cells. It is a highly conserved cellular structure that is responsible for the ATP-dependent proteolysis of most cellular proteins (Tanaka, Biochem Biophy. Res. Commun., 1998, 247, 537). The 26S proteasome consists of a 20S core catalytic complex that is capped at each end by a 19S regulatory subunit. The more complex eukaryotic 20S proteasome is composed of about 15 distinct 20-30 kDa subunits and is characterized by three major activities with respect to peptide substrates. For example, the proteasome displays tryptic-, chymotryptic-, and peptidylglutamyl peptide-hydrolytic activities (Rivett, Biochem. J., 1993, 291, 1 and Orlowski, Biochemistry, 1990, 29, 10289). Further, the proteasome has a unique active site mechanism which is believed to utilize a threonine residue as the catalytic nucleophile (See muller, et al., Science, 1995, 268, 579).

The proteasome is also required for activation of the transcription factor NF-κB by degradation of its inhibitory protein, IκB (Palombella, et al., Cell, 1994, 78, 773). NF-κB has a role in maintaining cell viability through the transcription of inhibitors of apoptosis. Blockade of NF-κB activity has been demonstrated to make cells more susceptible to apoptosis.

The 26S proteasome is able to degrade proteins that have been marked by the addition of ubiquitin molecules. Typically, ubiquitin is attached to the ε-amino groups of lysines in a multistep process utilizing ATP and E1 (ubiquitin activating) and E2 (ubiquitin-conjugating) enzymes. Multi-ubiquitinated substrate proteins are recognized by the 26S proteasome and are degraded. The multi-ubiquitin chains are generally released from the complex and ubiquitin is recycled (Goldberg, et al., Nature, 1992, 357, 375).

Numerous regulatory proteins are substrates for ubiquitin dependent proteolysis. Many of these proteins function as regulators of physiological as well as pathophysiological cellular processes. Alterations in proteasome activity have been implicated in a number of pathologies including neurodegenerative diseases such as Parkinson's disease, Alzheimer's disease, as well as occlusion/ischaemia reperfusion injuries, and aging of the central nervous system.

The invention includes compounds represented by formula I as illustrated below, or a pharmaceutically acceptable salt, ester or prodrug thereof: 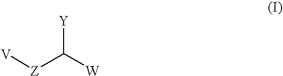
Wherein:
W is: 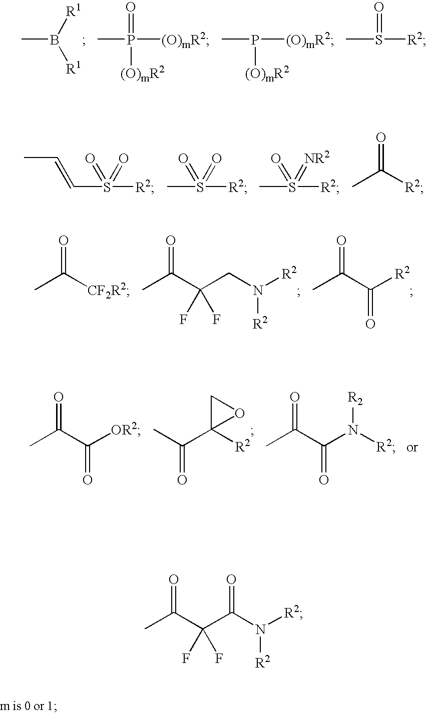
each R¹ is hydroxy, alkoxy, or aryloxy, or each R¹ is an oxygen atom and together with the boron, to which they are each bound, form a 5-7 membered monocylic, bicyclic, tricyclic or polycyclic ring, wherein the ring is optionally substituted with halogen, N, S, or O;
each R² is independently hydrogen, unsubstituted or substituted, saturated or unsaturated aliphatic, unsubstituted or substituted aryl, unsubstituted or substituted heteroaryl, unsubstituted or substituted cycloalkyl, or unsubstituted or substituted heterocycle; or two R² groups, which are bound to the same nitrogen atom, form together with that nitrogen atom, a 5-7 membered monocyclic heterocyclic ring system optionally substituted with halogen, N, S or O;
   Y is a substituted or unsubstituted, saturated or unsaturated aliphatic, unsubstituted or substituted cycloalkyl, unsubstituted or substituted aryl, unsubstituted or substituted heteroaryl;
   Z is selected from: 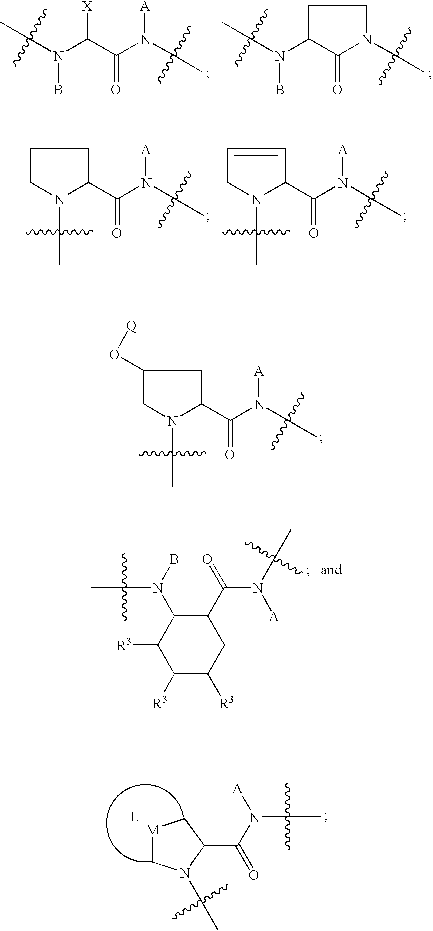
A and B are independently selected from hydrogen, and substituted or unsubstituted aliphatic;
X is a substituted or unsubstituted, saturated or unsaturated aliphatic, unsubstituted or substituted cycloalkyl, unsubstituted or substituted aryl, unsubstituted or substituted heteroaryl;
Q is a substituted or unsubstituted aryl, substituted or unsubstituted heteroaryl; or a substituted or unsubstituted, saturated or unsaturated aliphatic;
R³ are hydrogen; or two adjacent R³ are bound together to form substituted or unsubstituted aryl and the other R¹ is hydrogen;
   V is an acyl, a substituted or unsubstituted, saturated or unsaturated aliphatic, unsubstituted or substituted cycloalkyl, unsubstituted or substituted aryl, unsubstituted or substituted heteroaryl.

In one embodiment, the compound is not

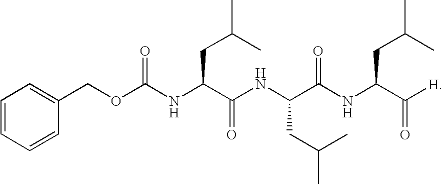

The composition for the treatment of spinal muscular atrophy (SMA) can comprise a peptide aldehyde. The structure of peptide aldehyde compounds can be illustrated below: 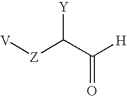
where V, Y and Z are as previously defined.

Peptide aldehydes have been reported to inhibit the chymotrypsin-like activity associated with the proteasome (Vinitsky, et al., Biochemistry, 1992, 31, 942:1; Tsubuki, et al., Biochem. Biophys. Res. Commun., 1993, 196, 1195; and Rock, et al., Cell, 1994, 78, 761). Dipeptidyl aldehyde inhibitors that have IC₅₀ values in the 10-100 nM range in vitro (Iqbal, M., et al., J. Med. Chem., 1995, 38, 2276) have also been reported. Stein, et al., U.S. Pat. No. 5,693,617 report peptidyl aldehyde compounds as proteasome inhibitors useful for reducing the rate of degradation of protein in an animal. Palombella, et al., WO 95/25533, report the use of peptide aldehydes to reduce the cellular content and activity of NF-κB in an animal by contacting cells of the animal with a peptide aldehyde inhibitor of proteasome function or ubiquitin conjugation.

The composition for the treatment of spinal muscular atrophy (SMA) can comprise a peptide vinyl sulfone. The structure of peptide vinyl sulfone compounds can be illustrated below: 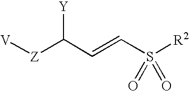
where V, Y Z and R² are as previously defined.

The composition for the treatment of spinal muscular atrophy (SMA) can comprise a peptide boronate. The structure of peptide boronate compounds can be illustrated below: 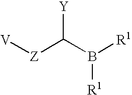
where V, Y Z and R¹ are as previously defined.

In a preferred embodiment, the peptide boronate is bortezomib, sold under the trademark Velcade®. N-terminal peptidyl boronic ester and acid compounds have been reported previously (U.S. Pat. Nos. 4,499,082 and 4,537,773; WO 91/13904; Kettner, et al., J. Biol. Chem., 1984, 259(24), 15106). These compounds are reported to be inhibitors of certain proteolytic enzymes. WO 96/13266 report boronic ester and acid compounds and a method for reducing the rate of degradation of proteins. Pharmaceutically acceptable compositions of boronic acids and novel boronic acid anhydrides and boronate ester compounds are reported by Plamondon, et al., U.S. Patent Application Pub. No. 2002/0188100. A series of di- and tripeptidyl boronic acids are shown to be inhibitors of 20S and 26S proteasome in Gardner, et al., Biochem. J., 2000, 346, 447. Other boron-containing peptidyl and related compounds are reported in U.S. Pat. Nos. 5,250,720; 5,242,904; 5,187,157; 5,159,060; 5,106,948; 4,963,655; 4,499,082; and WO 89/09225, WO/98/17679, WO 98/22496, WO 00/66557, WO 02/059130, WO 03/15706, WO 96/12499, WO 95/20603, WO 95/09838, WO 94/25051, WO 94/25049, WO 94/04653, WO 02/08187, EP 632026, and EP 354522.

The composition for the treatment of spinal muscular atrophy (SMA) can comprise a peptide epoxiketone. The structure of peptide epoxiketone compounds can be illustrated below: 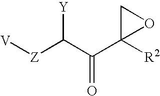
where V, Y Z and R² are as previously defined. Preferably, the peptide epoxiketones are epoxomicin and eponemycin.

The compounds can also be represented by lactams and β-lactones of formulae II and III as illustrated below, or a pharmaceutically acceptable salt, ester or prodrug thereof. 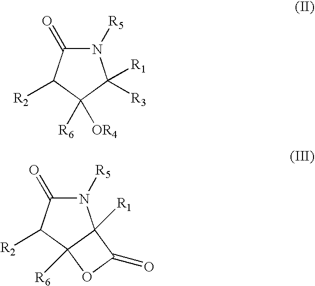
Wherein:
R₁, R₂ and R₆ are independently selected from hydrogen, substituted or unsubstituted, saturated or unsaturated aliphatic;
R₃ is an acyl, a substituted or unsubstituted, saturated or unsaturated aliphatic;
R₄ and R₅ are independently selected from hydrogen and substituted or unsubstituted aliphatic.

In a preferred embodiment, the compounds of formulae II and III are lactacystin, omuralide, and antiprotealide. Lactacystin is a Streptomyces metabolite that specifically inhibits the proteolytic activity of the proteasome complex (Fenteany, et al., Science, 1995, 268, 726). This molecule is capable of inhibiting the proliferation of several cell types (Fenteany, et al., Proc. Natl. Acad. Sci. USA, 1994, 91, 3358). It has been shown that lactacystin binds irreversibly, through its β-lactone moiety, to a threonine residue located at the amino terminus of the β-subunit of the proteasome.

Other inhibitors include α-ketoamide compounds useful for treating disorders mediated by 20S proteasome in mammals are reported in Wang et al., U.S. Pat. No. 6,075,150. France, et al., WO 00/64863, report the use of 2,4-diamino-3-hydroxycarboxylic acid derivatives as proteasome inhibitors. Carboxylic acid derivatives as proteasome inhibitors are reported by Yamaguchi et al., EP 1166781. Ditzel, et al., EP 0 995 757 report bivalent inhibitors of the proteasome. 2-Aminobenzylstatine derivatives that inhibit non-covalently the chymotrypsin-like activity of the 20S proteasome have been reported by Garcia-Echeverria, et al., Bioorg. Med. Chem. Lett., 2001, 11, 1317. Inhibition of the 26S and 20S proteasome by indanone derivatives and a method for inhibiting cell proliferation using indanone derivatives are reported by Lum et al., U.S. Pat. No. 5,834,487.

All of the above references and patents are incorporated herein by reference in their entirety.

Definitions

Listed below are definitions of various terms used to describe this invention. These definitions apply to the terms as they are used throughout this specification and claims, unless otherwise limited in specific instances, either individually or as part of a larger group.

As used herein, “full length SMN gene expression” or “expression level of SMN exon 7” refers to a scenario where an SMN gene is transcribed and the resulting transcripts contain exon 7 of an SMN gene. Specifically, it is of no consequence whether the exon 7-containing transcript is transcribed from the human SMN1 gene or from the human SMN2 gene. Transcripts containing SMN exon 7 are translated into the 294 amino acid SMN polypeptide. The amino acid sequence of the 294 amino acid SMN polypeptide is described in GenBank entry “GI:624186.” The nucleic acid sequence of SMN exon 7 is the sequence contained between nucleotides about 868 and about 921 of GenBank entry “GI:624185.” The identify of the sixth base of exon 7 can be C (cytosine) if the transcript is derived from SMN1 or U (uracil) if the transcript is derived from SMN2. Exon 7 expression can be analyzed in cells in which SMN1 is deleted or mutated. Thus, the relevant SMN exon 7 sequence contains a uracil at position 873 while the remainder of the sequence is as recited from nucleotides about 868 to about 921 of GenBank entry “GI:624185.”

The term “aryl,” as used herein, refers to a mono- or polycyclic carbocyclic ring system having one or two aromatic rings including, but not limited to, phenyl, naphthyl, tetrahydronaphthyl, indanyl, idenyl and the like.

The term “heteroaryl,” as used herein, refers to a mono- or polycyclic (e.g. bi-, or tri-cyclic or more) aromatic radical or ring having from five to ten ring atoms of which one or more ring atom is selected from, for example, S, O and N; zero, one or two ring atoms are additional heteroatoms independently selected from, for example, S, O and N; and the remaining ring atoms are carbon, wherein any N or S contained within the ring may be optionally oxidized. Heteroaryl includes, but is not limited to, pyridinyl, pyrazinyl, pyrimidinyl, pyrrolyl, pyrazolyl, imidazolyl, thiazolyl, oxazolyl, isooxazolyl, thiadiazolyl, oxadiazolyl, thiophenyl, furanyl, quinolinyl, isoquinolinyl, benzimidazolyl, benzooxazolyl, quinoxalinyl, and the like.

In accordance with the invention, any of the aryls, substituted aryls, heteroaryls and substituted heteroaryls described herein, can be any aromatic group. Aromatic groups can be substituted or unsubstituted.

An “aliphatic group” is non-aromatic moiety that may contain any combination of carbon atoms, hydrogen atoms, halogen atoms, oxygen, nitrogen or other atoms, and optionally contain one or more units of unsaturation, e.g., double and/or triple bonds. An aliphatic group may be straight chained, branched or cyclic and preferably contains between about 1 and about 24 carbon atoms, more typically between about 1 and about 12 carbon atoms. In addition to aliphatic hydrocarbon groups, aliphatic groups include, for example, polyalkoxyalkyls, such as polyalkylene glycols, polyamines, and polyimines, for example. Such aliphatic groups may be further substituted.

The term “alicyclic,” as used herein, denotes a monovalent group derived from a monocyclic or bicyclic saturated carbocyclic ring compound by the removal of a single hydrogen atom. Examples include, but not limited to, cyclopropyl, cyclobutyl, cyclopentyl, cyclohexyl, bicyclo[2.2.1]heptyl, and bicyclo[2.2.2]octyl. Such alicyclic groups may be further substituted.

The term “heterocyclic” as used herein, refers to a non-aromatic 5-, 6- or 7-membered ring or a bi- or tri-cyclic group fused system, where (i) each ring contains between one and three heteroatoms independently selected from oxygen, sulfur and nitrogen, (ii) each 5-membered ring has 0 to I double bonds and each 6-membered ring has 0 to 2 double bonds, (iii) the nitrogen and sulfur heteroatoms may optionally be oxidized, (iv) the nitrogen heteroatom may optionally be quaternized, (iv) any of the above rings may be fused to a benzene ring, and (v) the remaining ring atoms are carbon atoms which may be optionally oxo-substituted. Representative heterocycloalkyl groups include, but are not limited to, [1,3]dioxolane, pyrrolidinyl, pyrazolinyl, pyrazolidinyl, imidazolinyl, imidazolidinyl, piperidinyl, piperazinyl, oxazolidinyl, isoxazolidinyl, morpholinyl, thiazolidinyl, isothiazolidinyl, quinoxalinyl, pyridazinonyl, and tetrahydrofuryl. Such heterocyclic groups may be further substituted.

The terms “substituted aryl’, “substituted heteroaryl, or “substituted aliphatic,” as used herein, refer to aryl, heteroaryl, aliphatic groups as previously defined, substituted by independent replacement of one, two, or three or more of the hydrogen atoms thereon with substituents including, but not limited to, —F, —Cl, —Br, —I, —OH, protected hydroxyl, —NO₂, —CN, —C₁-C₁₂-alkyl optionally substituted with, for example, halogen, C₂-C₁₂-alkenyl optionally substituted with, for example, halogen, —C₂-C₁₂-alkynyl optionally substituted with, for example, halogen, —NH₂, protected amino, —NH—C₁-C₁₂-alkyl, —NH—C₂-C₁₂-alkenyl, —NH—C₂-C₁₂-alkenyl, —NH—C₃-C₁₂-cycloalkyl, —NH -aryl, —NH -heteroaryl, —NH -heterocycloalkyl, -dialkylamino, -diarylamino, -diheteroarylamino, —O—C₁-C₁₂-alkyl, —O—C₂-C₁₂-alkenyl, —O—C₂-C₁₂-alkenyl, —O—C₃-C₁₂-cycloalkyl, —O-aryl, —O-heteroaryl, —O-heterocycloalkyl, —C(O)—C₁-C₁₂-alkyl, —C(O)—C₂-C₁₂-alkenyl, —C(O)—C₂-C₁₂-alkenyl, —C(O)—C₃-C₁₂-cycloalkyl, —C(O)-aryl, —C(O)—heteroaryl, —C(O)-heterocycloalkyl, —CONH₂, —CONH—C₁-C₁₂-alkyl, —CONH—C₂-C₁₂-alkenyl, —CONH—C₂-C₁₂-alkenyl, —CONH—C₃-C₁₂-cycloalkyl, —CONH-aryl, —CONH—heteroaryl, —CONH-heterocycloalkyl, —OCO₂—C₁-C₁₂-alkyl, —OCO₂—C₂-C₁₂-alkenyl, —OCO₂—C₂-C₁₂-alkenyl, —OCO₂—C₃-C₁₂-cycloalkyl, —OCO₂-aryl, —OCO₂-heteroaryl, —OCO₂-heterocycloalkyl, —OCONH₂, —OCONH—C₁-C₁₂-alkyl, —OCONH—C₂-C₁₂-alkenyl, —OCONH—C₂-C₁₂-alkenyl, —OCONH—C₃-C₁₂-cycloalkyl, —OCONH— aryl, —OCONH—heteroaryl, —OCONH— heterocycloalkyl, —NHC(O)—C₁-C₁₂-alkyl, —NHC(O)—C₂-C₁₂-alkenyl, —NHC(O)—C₂-C₁₂-alkenyl, —NHC(O)—C₃-C₁₂-cycloalkyl, —NHC(O)-aryl, —NHC(O)-heteroaryl, —NHC(O)-heterocycloalkyl, —NHCO₂—C₁-C₁₂-alkyl, —NHCO₂—C₂-C₁₂-alkenyl, —NHCO₂—C₂-C₁₂-alkenyl, —NHCO₂—C₃-C₁₂-cycloalkyl, —NHCO₂— aryl, —NHCO₂-heteroaryl, —NHCO₂— heterocycloalkyl, —NHC(O)NH₂, —NHC(O)NH—C₁-C₁₂-alkyl, —NHC(O)NH—C₂-C₁₂-alkenyl, —NHC(O)NH—C₂-C₁₂-alkenyl, —NHC(O)NH—C₃-C₁₂-cycloalkyl, —NHC(O)NH-aryl, —NHC(O)NH-heteroaryl, —NHC(O)NH-heterocycloalkyl, NHC(S)NH₂, —NHC(S)NH—C₁-C₁₂-alkyl, —NHC(S)NH—C₂-C₁₂-alkenyl, —NHC(S)NH—C₂-C₁₂-alkenyl, —NHC(S)NH—C₃-C₁₂-cycloalkyl, —NHC(S)NH-aryl, —NHC(S)NH—heteroaryl, —NHC(S)NH-heterocycloalkyl, —NHC(NH)NH₂, —NHC(NH)NH—C₁-C₁₂-alkyl, —NHC(NH)NH-C₂-C₁₂-alkenyl, —NHC(NH)NH—C₂-C₁₂-alkenyl, —NHC(NH)NH—C₃-C₁₂-cycloalkyl, —NHC(NH)NH-aryl, —NHC(NH)NH-heteroaryl, —NHC(NH)NH—heterocycloalkyl, —NHC(NH)—C₁-C₁₂-alkyl, —NHC(NH)—C₂-C₁₂-alkenyl, —NHC(NH)—C₂-C₁₂-alkenyl, —NHC(NH)—C₃-C₁₂-cycloalkyl, —NHC(NH)-aryl, —NHC(NH)-heteroaryl, —NHC(NH)-heterocycloalkyl, —C(NH)NH—C₁-C₁₂-alkyl, —C(NH)NH—C₂-C₁₂-alkenyl, —C(NH)NH—C₂-C₂-alkenyl, —C(NH)NH—C₃-C₂-cycloalkyl, —C(NH)NH-aryl, —C(NH)NH—heteroaryl, —C(NH)NH-heterocycloalkyl, —S(O)—C₁-C₂-alkyl, —S(O)—C₂-C₁₂-alkenyl, —S(O)—C₂-C₁₂-alkenyl, —S(O)—C₃-C₁₂-cycloalkyl, —S(O)-aryl, —S(O)-heteroaryl, —S(O)—heterocycloalkyl —SO₂NH₂, —SO₂NH—C₁-C₁₂-alkyl, —SO₂NH—C₂-C₁₂-alkenyl, —SO₂NH—C₂-C₁₂-alkenyl, —SO₂NH—C₃-C₁₂-cycloalkyl, —SO₂NH— aryl, —SO₂NH— heteroaryl, —SO₂NH-heterocycloalkyl, —NHSO₂—C₁-C₁₂-alkyl, —NHSO₂—C₂-C₁₂-alkenyl, —NHSO₂—C₂-C₁₂-alkenyl, —NHSO₂—C₃-C₁₂-cycloalkyl, —NHSO₂-aryl, —NHSO₂-heteroaryl, —NHSO₂-heterocycloalkyl, —CH₂NH₂, —CH₂SO₂CH₃, -aryl, -arylalkyl, -heteroaryl, -heteroarylalkyl, -heterocycloalkyl, —C₃-C₁₂-cycloalkyl, polyalkoxyalkyl, polyalkoxy, -methoxymethoxy, -methoxyethoxy, —SH, —S-C₁-C₁₂-alkyl, —S—C₂-C₁₂-alkenyl, —S—C₂-C₁₂-alkenyl, —S—C₃-C₁₂-cycloalkyl, —S-aryl, —S-heteroaryl, —S-heterocycloalkyl, or methylthiomethyl. It is understood that the aryls, heteroaryls, alkyls, and the like can be further substituted.

The term “halogen,” as used herein, refers to an atom selected from fluorine, chlorine, bromine and iodine.

Combinations of substituents and variables envisioned by this invention are only those that result in the formation of stable compounds. The term “stable”, as used herein, refers to compounds which possess stability sufficient to allow manufacture and which maintains the integrity of the compound for a sufficient period of time to be useful for the purposes detailed herein (e.g., therapeutic or prophylacetic administration to a subject).

The synthesized compounds can be separated from a reaction mixture and further purified by a method such as column chromatography, high pressure liquid chromatography, or recrystallization. As can be appreciated by the skilled artisan, further methods of synthesizing the compounds of the formulae herein will be evident to those of ordinary skill in the art. Additionally, the various synthetic steps may be performed in an alternate sequence or order to give the desired compounds. Synthetic chemistry transformations and protecting group methodologies (protection and deprotection) useful in synthesizing the compounds described herein are known in the art and include, for example, those such as described In R. Larock, Comprehensive Organic Transformations, VCH Publishers (1989); T. W. Greene and P. G. M. Wuts, Protective Groups in Organic Synthesis, 2d. Ed., John Wiley and Sons (1991); L. Fieser and M. Fieser, Fieser and Fieser's Reagents for Organic Synthesis, John Wiley and Sons (1994); and L. Paquette, ed., Encyclopedia of Reagents for Organic Synthesis, John Wiley and Sons (1995), and subsequent editions thereof.

The term “subject” as used herein refers to an animal. Preferably the animal is a mammal. More preferably the mammal is a human. A subject also refers to, for example, dogs, cats, horses, cows, pigs, guinea pigs, birds and the like and, include in particular, animal models for SMA.

The compounds of this invention may be modified by appending appropriate functionalities to enhance selective biological properties. Such modifications are known in the art and may include those which increase biological penetration into a given biological system (e.g., blood, lymphatic system, central nervous system), increase oral availability, increase solubility to allow administration by injection, alter metabolism and alter rate of excretion.

The compounds described herein contain one or more asymmetric centers and thus give rise to enantiomers, diastereomers, and other stereoisomeric forms that may be defined, in terms of absolute stereochemistry, as (R)- or (S)-, or as (D)- or (L)- for amino acids. The present invention is meant to include all such possible isomers, as well as their racemic and optically pure forms. Optical isomers may be prepared from their respective optically active precursors by the procedures described above, or by resolving the racemic mixtures. The resolution can be carried out in the presence of a resolving agent, by chromatography or by repeated crystallization or by some combination of these techniques which are known to those skilled in the art. Further details regarding resolutions can be found in Jacques, et al., Enantiomers, Racemates, and Resolutions (John-Wiley & Sons, 1981). When the compounds described herein contain olefinic double bonds, other unsaturation, or other centers of geometric asymmetry, and unless specified otherwise, it is intended that the compounds include both E and Z geometric isomers or cis- and trans-isomers. Likewise, all tautomeric forms are also intended to be included. The configuration of any carbon-carbon double bond appearing herein is selected for convenience only and is not intended to designate a particular configuration unless the text so states; thus a carbon-carbon double bond or carbon-heteroatom double bond depicted arbitrarily herein as trans may be cis, trans, or a mixture of the two in any proportion.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge, et al. describes pharmaceutically acceptable salts in detail in J. Pharmaceutical Sciences, 66: 1-19 (1977). The salts can be prepared in situ during the final isolation and purification of the compounds of the invention, or separately by reacting the free base function with a suitable organic acid. Examples of pharmaceutically acceptable include, but are not limited to, nontoxic acid addition salts are salts of an amino group formed with inorganic acids such as hydrochloric acid, hydrobromic acid, phosphoric acid, sulfuric acid and perchloric acid or with organic acids such as acetic acid, maleic acid, tartaric acid, citric acid, succinic acid or malonic acid or by using other methods used in the art such as ion exchange. Other pharmaceutically acceptable salts include, but are not limited to, adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, alkyl having from 1 to 6 carbon atoms, sulfonate and aryl sulfonate.

As used herein, the term “pharmaceutically acceptable ester” refers to esters which hydrolyze in vivo and include those that break down readily in the human body to leave the parent compound or a salt thereof. Suitable ester groups include, for example, those derived from pharmaceutically acceptable aliphatic carboxylic acids, particularly alkanoic, alkenoic, cycloalkanoic and alkanedioic acids, in which each alkyl or alkenyl moiety advantageously has not more than 6 carbon atoms. Examples of particular esters include, but are not limited to, formates, acetates, propionates, butyrates, acrylates and ethylsuccinates.

The term “pharmaceutically acceptable prodrugs” as used herein refers to those prodrugs of the compounds of the present invention which are, within the scope of sound medical judgment, suitable for use in contact with the tissues of humans and lower animals with undue toxicity, irritation, allergic response, and the like, commensurate with a reasonable benefit/risk ratio, and effective for their intended use, as well as the zwitterionic forms, where possible, of the compounds of the present invention. “Prodrug”, as used herein means a compound which is convertible in vivo by metabolic means (e.g. by hydrolysis) to a compound of Formula I. Various forms of prodrugs are known in the art, for example, as discussed in Bundgaard, (ed.), Design of Prodrugs, Elsevier (1985); Widder, et al. (ed.), Methods in Enzymology, vol. 4, Academic Press (1985); Krogsgaard-Larsen, et al., (ed). “Design and Application of Prodrugs, Textbook of Drug Design and Development, Chapter 5, 113-191 (1991); Bundgaard, et al., Journal of Drug Deliver Reviews, 8:1-38(1992); Bundgaard, J. of Pharmaceutical Sciences, 77:285 et seq. (1988); Higuchi and Stella (eds.) Prodrugs as Novel Drug Delivery Systems, American Chemical Society (1975); and Bernard Testa & Joachim Mayer, “Hydrolysis In Drug And Prodrug Metabolism: Chemistry, Biochemistry And Enzymology,” John Wiley and Sons, Ltd. (2002).

Pharmaceutical Compositions.

The pharmaceutical compositions of the present invention comprise a therapeutically effective amount of a compound of the present invention formulated together with one or more pharmaceutically acceptable carriers or excipients.

As used herein, the term “pharmaceutically acceptable carrier or excipient” means a non-toxic, inert solid, semi-solid or liquid filler, diluent, encapsulating material or formulation auxiliary of any type. Some examples of materials which can serve as pharmaceutically acceptable carriers are sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and suppository waxes; oils such as peanut oil, cottonseed oil, safflower oil, sesame oil, olive oil, corn oil and soybean oil; glycols such as propylene glycol; esters such as ethyl oleate and ethyl laurate; agar; buffering agents such as magnesium hydroxide and aluminun hydroxide; alginic acid; pyrogen-free water; isotonic saline; Ringer's solution; ethyl alcohol, and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator.

The pharmaceutical compositions of this invention may be administered orally, parenterally, by inhalation, topically, rectally, nasally, buccally, vaginally or via an implanted reservoir, preferably by oral administration or administration by injection. The pharmaceutical compositions of this invention may contain any conventional non-toxic pharmaceutically-acceptable carriers, adjuvants or vehicles. In some cases, the pH of the formulation may be adjusted with pharmaceutically acceptable acids, bases or buffers to enhance the stability of the formulated compound or its delivery form. The term parenteral as used herein includes subcutaneous, intracutaneous, intravenous, intramuscular, intraarticular, intraarterial, intrasynovial, intrasternal, intrathecal, intralesional and intracranial injection or infusion techniques. By pharmaceutically acceptable formulation is meant, a composition or formulation that allows for the effective distribution of the compounds of the instant invention in the physical location most suitable for their desired activity. In some embodiments, compounds, e.g., P-glycoprotein inhibitors (such as Pluronic P85), which can enhance entry of drugs into the CNS (Jolliet-Riant and Tillement, 1999, Fundam. Clin. Pharmacol., 13, 16-26) can be included and nanoparticles, such as those made of polybutylcyanoacrylate, which can deliver drugs across the blood brain barrier and can alter neuronal uptake mechanisms (Prog Neuropsychopharmacol Biol Psychiatry, 23, 941-949, 1999). The use of a liposome or other drug carrier comprising the proteasome inhibitors of the instant invention can potentially localize the drug, for example, in certain tissue types, such as the tissues of the central nervous system. A liposome formulation which can facilitate the association of drug with active transport molecules on the surface of the blood brain barrier, such as, the mannose and galactose transporter is also useful. This approach may provide enhanced delivery of the drug to central nervous system cells by taking advantage of the efficiency of the transporters to deliver sugars to the brain. Other non-limiting examples of delivery strategies for the proteasome inhibitors of the instant invention include material described in Boado et al., 1998, J. Pharm. Sci., 87, 1308-1315; Tyler et al., 1999, FEBS Lett., 421, 280-284; Pardridge et al., 1995, PNAS USA., 92, 5592-5596; Boado, 1995, Adv. Drug Delivery Rev., 15, 73-107; Aldrian-Herrada et al., 1998, Nucleic Acids Res., 26, 4910-4916; and Tyler et al., 1999, PNAS USA., 96, 7053-7058. The preferred method for targeting the nervous system, such as spinal cord glia, is by intrathecal delivery. The targeted inhibitor is released into the surrounding CSF and/or tissues and the released inhibitors can penetrate into the spinal cord parenchyma, just after acute intrathecal injections. For a comprehensive review on drug delivery strategies including CNS delivery, see Ho et al., 1999, Curr. Opin. Mol. Ther., 1, 336-343 and Jain, Drug Delivery Systems: Technologies and Commercial Opportunities, Decision Resources, 1998 and Groothuis et al., 1997, J Neuro Virol., 3, 387-400.

Liquid dosage forms for oral administration include pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable preparations, for example, sterile injectable aqueous or oleaginous suspensions, may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables.

The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a drug, it is often desirable to slow the absorption of the drug from subcutaneous or intramuscular injection. This may be accomplished by the use of a liquid suspension of crystalline or amorphous material with poor water solubility. The rate of absorption of the drug then depends upon its rate of dissolution, which, in turn, may depend upon crystal size and crystalline form. Alternatively, delayed absorption of a parenterally administered drug form is accomplished by dissolving or suspending the drug in an oil vehicle. Injectable depot forms are made by forming microencapsule matrices of the drug in biodegradable polymers such as polylactide-polyglycolide. Depending upon the ratio of drug to polymer and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions that are compatible with body tissues.

Compositions for rectal or vaginal administration are preferably suppositories which can be prepared by mixing the compounds of this invention with suitable non-irritating excipients or carriers such as cocoa butter, polyethylene glycol or a suppository wax which are solid at ambient temperature but liquid at body temperature and therefore melt in the rectum or vaginal cavity and release the active compound.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or: a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like.

The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes.

Dosage forms for topical or transdermal administration of a compound of this invention include ointments, pastes, creams, lotions, gels, powders, solutions, sprays, inhalants or patches. The active component is admixed under sterile conditions with a pharmaceutically acceptable carrier and any needed preservatives or buffers as may be required. Ophthalmic formulation, ear drops, eye ointments, powders and solutions are also contemplated as being within the scope of this invention.

The ointments, pastes, creams and gels may contain, in addition to an active compound of this invention, excipients such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to the compounds of this invention, excipients such as lactose, talc, silicic acid, aluminum hydroxide, calcium silicates and polyamide powder, or mixtures of these substances. Sprays can additionally contain customary propellants such as chlorofluorohydrocarbons.

Transdermal patches have the added advantage of providing controlled delivery of a compound to the body. Such dosage forms can be made by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

For pulmonary delivery, a therapeutic composition of the invention is formulated and administered to the patient in solid or liquid particulate form by direct administration e.g., inhalation into the respiratory system. Solid or liquid particulate forms of the active compound prepared for practicing the present invention include particles of respirable size: that is, particles of a size sufficiently small to pass through the mouth and larynx upon inhalation and into the bronchi and alveoli of the lungs. Delivery of aerosolized therapeutics, particularly aerosolized antibiotics, is known in the art (see, for example U.S. Pat. No. 5,767,068 to VanDevanter et al., U.S. Pat. No. 5,508,269 to Smith et al., and WO 98/43,650 by Montgomery, all of which are incorporated herein by reference). A discussion of pulmonary delivery of antibiotics is also found in U.S. Pat. No. 6,014,969, incorporated herein by reference.

By a “therapeutically effective amount” of a compound of the invention is meant an amount of the compound which confers a therapeutic effect on the treated subject, at a reasonable benefit/risk ratio applicable to any medical treatment. The therapeutic effect may be objective (i.e., measurable by some test or marker) or subjective (i.e., subject gives an indication of or feels an effect). An effective amount of the compound described above may range from about 0.1 mg/Kg to about 500 mg/Kg, preferably from about 1 to about 50 mg/Kg. Effective doses will also vary depending on route of administration, as well as the possibility of co-usage with other agents. It will be understood, however, that the total daily usage of the compounds and compositions of the present invention will be decided by the attending physician within the scope of sound medical judgment. The specific therapeutically effective dose level for any particular patient will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or contemporaneously with the specific compound employed; and like factors well known in the medical arts.

The total daily dose of the compounds of this invention administered to a human or other animal in single or in divided doses can be in amounts, for example, from 0.01 to 50 mg/kg body weight or more usually from 0.1 to 25 mg/kg body weight. Single dose compositions may contain such amounts or submultiples thereof to make up the daily dose. In general, treatment regimens according to the present invention comprise administration to a patient in need of such treatment from about 10 mg to about 1000 mg of the compound(s) of this invention per day in single or multiple doses.

The methods herein contemplate administration of an effective amount of compound or compound composition to achieve the desired or stated effect. Typically, the pharmaceutical compositions of this invention will be administered from about 1 to about 6 times per day or alternatively, as a continuous infusion. Such administration can be used as a chronic or acute therapy. The amount of active ingredient that may be combined with pharmaceutically excipients or carriers to produce a single dosage form will vary depending upon the host treated and the particular mode of administration. A typical preparation will contain from about 5% to about 95% active compound (w/w). Alternatively, such preparations may contain from about 20% to about 80% active compound.

Lower or higher doses than those recited above may be required. Specific dosage and treatment regimens for any particular patient will depend upon a variety of factors, including the activity of the specific compound employed, the age, body weight, general health status, sex, diet, time of administration, rate of excretion, drug combination, the severity and course of the disease, condition or symptoms, the patient's disposition to the disease, condition or symptoms, and the judgment of the treating physician.

Upon improvement of a patient's condition, a maintenance dose of a compound, composition or combination of this invention may be administered, if necessary. Subsequently, the dosage or frequency of administration, or both, may be reduced, as a function of the symptoms, to a level at which the improved condition is retained when the symptoms have been alleviated to the desired level. Patients may, however, require intermittent treatment on a long-term basis upon any recurrence of disease symptoms.

When the compositions of this invention comprise a combination of a compound of the formulae described herein and one or more additional therapeutic or prophylacetic agents, both the compound and the additional agent should be present at dosage levels of between about 1 to 100%, and more preferably between about 5 to 95% of the dosage normally administered in a monotherapy regimen. The additional agents may be administered separately, as part of a multiple dose regimen, from the compounds of this invention. Alternatively, those agents may be part of a single dosage form, mixed together with the compounds of this invention in a single composition.

The invention is further related to the appreciation that the assays described herein can be efficiently used as the primary assay for selecting a candidate for the treatment of SMA. Thus, the invention relates to a method for selecting a candidate for the treatment of SM-A comprising (a) contacting the candidate with a fibroblast cell culture derived from an SMA patient under conditions and for a period of time sufficient for SMN protein expression and gem formation; (b) determining the formation of gems of SMN protein; and (c) selecting the candidate. The formation of gems can be determined by establishing the percentage of fibroblasts with gems in the cell culture. In other embodiments, the number of gems or concentration of gems in the culture can be determined.

Cell lines are derived from SMA patients. Such cells are termed “SMA cells” herein. The cells are isolated from a variety of sources and tissues. For example, the cells can be isolated from a blood sample or from a biopsy. The cell can be a stem cell, a fibroblast, a neuronal cell or a lymphoid cell. The cells can be propagated in culture according to cell type and origin of the cells. The requisite growth factors can be provided in the media. For example, the media can be supplemented with fetal calf serum, a cocktail of purified factors, or an individual growth factor. The cells can be propagated without being immortalized. Alternatively, the cells can immortalized using a virus or a plasmid bearing an oncogene, or a transforming viral protein, e.g., papilloma E6 or E7 protein. The source of the fibroblasts for cell culture can be isolated from a patient with SMA or derived from such an isolation. In one embodiment, the cells are a clonal cell culture derived from an SMA patient.

Procedures for isolating and maintaining cells lines are well known in the art and can be found in suitable laboratory manuals. The cells can be grown in sufficient amount to screen an array of test compounds. Alternatively, cells can be used to assess the effectiveness of individual compounds as SMA treatments. Equivalent cell culture conditions can also be used. Conditions can be considered “equivalent” if SMN protein and gem formation are achieved in the presence of a known proteasome inhibitor, such as those exemplified herein, yet, in the absence of such inhibitor, substantially less SMN protein and gem formation are achieved, thereby providing a meaningful basis for comparison.

The candidate that is selected preferably establishes a percentage of about 50% or more fibroblasts with gems in the cell culture at a concentration of about 10 uM. This is not to suggest that the conditions of the assay must be those set forth herein. Because equivalent culture conditions can be readily envisioned and modifying such conditions can have an impact on the qualitative results of the culture, the selection of numerical values that will meet all culture conditions is impractical. However, a person of ordinary skill in the art can determine the relative, or equivalent, activity of a candidate under a given set of culture conditions based upon the culture conditions exemplified herein.

The candidate selected according to the present method preferably establishes a percentage of about 50% or more fibroblasts with gems in the cell culture at a concentration of about 1 uM, such as about 0.5 uM, such as 0.1 uM, under said conditions.

The invention further relates to compounds, particularly proteasome inhibitors, selected by such a method and methods of treating spinal muscular atrophy (SMA) comprising administering a therapeutically effective amount of such compounds.

Unless otherwise defined, all technical and scientific terms used herein are accorded the meaning commonly known to one of ordinary skill in the art. All publications, patents, published patent applications, and other references mentioned herein are hereby incorporated by reference in their entirety. The embodiments of the invention should not be deemed to be mutually exclusive and can be combined.

What is claimed is:

1. A method of treating spinal muscular atrophy (SMA) comprising administering a therapeutically effective amount of at least one proteasome inhibitor or a pharmaceutically acceptable salt, isomer, prodrug, analog, metabolite or derivative thereof.

2. The method of claim 1, wherein the level of gemini of coiled bodies (gems) of SMN protein are increased.

3. The method of claim 1, wherein said proteasome inhibitor is selected from the group consisting of peptide aldehydes, peptide vinyl sulfones, peptide boronates, peptide epoxiketones, β-lactones or a pharmaceutically acceptable salt, isomer, prodrug, analog, metabolite or derivative thereof.

4. The method of claim 3, wherein said proteasome inhibitor is bortezomib (Velcade®).

5. The method of claim 3, wherein said proteasome inhibitor is lactacystin.

6. The method of claim 3, wherein said proteasome inhibitor is omuralide.

7. The method of claim 3, wherein said proteasome inhibitor is antiprotealide.

8. The method of claim 3, wherein said proteasome inhibitor is epoxomicin.

9. The method of claim 3, wherein said proteasome inhibitor is eponemycin.

10. The method of claim 1, wherein said proteasome inhibitor of the present invention is represented by formula (I): 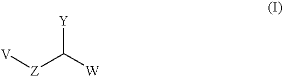
Wherein:
W is: 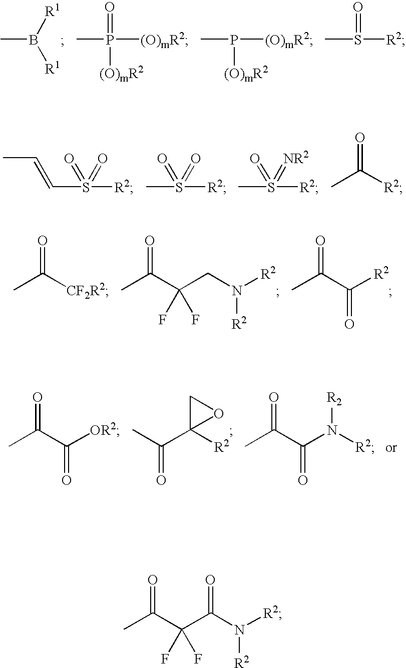
each R¹ is hydroxy, alkoxy, or aryloxy, or each R¹ is an oxygen atom and together with the boron, to which they are each bound, form a 5-7 membered monocylic, bicyclic, tricyclic or polycyclic ring, wherein the ring is optionally substituted with halogen, N, S, or O;
each R² is independently hydrogen, unsubstituted or substituted, saturated or unsaturated aliphatic, unsubstituted or substituted aryl, unsubstituted or substituted heteroaryl, unsubstituted or substituted cycloalkyl, or unsubstituted or substituted heterocycle; or two R² groups, which are bound to the same nitrogen atom, form together with that nitrogen atom, a 5-7 membered monocyclic heterocyclic ring system optionally substituted with halogen, N, S or O;
Y is a substituted or unsubstituted, saturated or unsaturated aliphatic, unsubstituted or substituted cycloalkyl, unsubstituted or substituted aryl, unsubstituted or substituted heteroaryl;
Z is selected from: 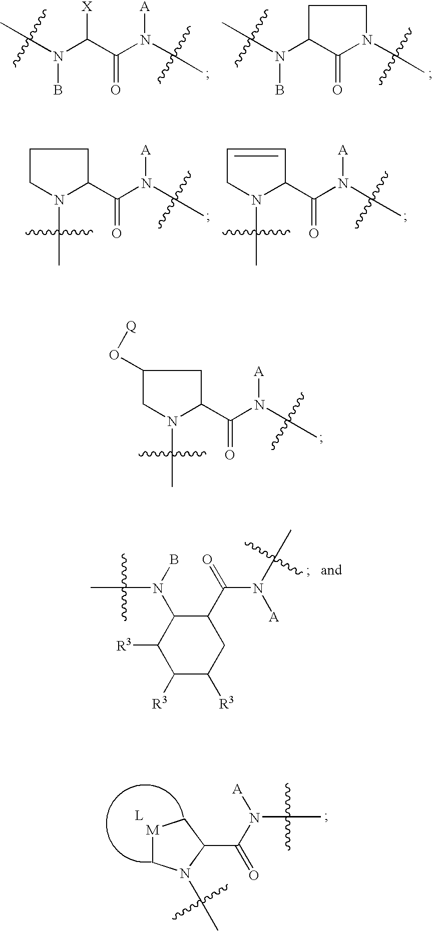
A and B are independently selected from hydrogen, and substituted or unsubstituted aliphatic;
X is a substituted or unsubstituted, saturated or unsaturated aliphatic, unsubstituted or substituted cycloalkyl, unsubstituted or substituted aryl, unsubstituted or substituted heteroaryl;
Q is a substituted or unsubstituted aryl, substituted or unsubstituted heteroaryl;
or a substituted or unsubstituted, saturated or unsaturated aliphatic;
R³ are hydrogen; or two adjacent R³ are bound together to form substituted or unsubstituted aryl and the other R³ is hydrogen;
V is an acyl, a substituted or unsubstituted, saturated or unsaturated aliphatic, unsubstituted or substituted cycloalkyl, unsubstituted or substituted aryl, unsubstituted or substituted heteroaryl;
with the proviso that formula (I) is not

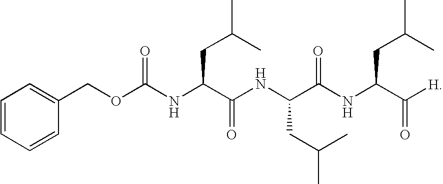

11. The method of claim 1, wherein said proteasome inhibitor of the present invention is represented by formulae (II) and (III): 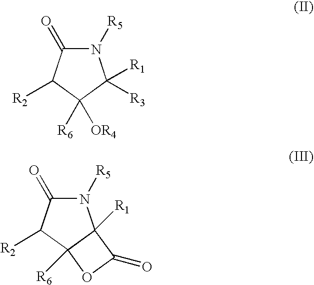
Wherein:
R₁, R₂ and R₆ are independently selected from hydrogen, substituted or unsubstituted, saturated or unsaturated aliphatic;
R₃ is an acyl, a substituted or unsubstituted, saturated or unsaturated aliphatic;
R₄ and R₅ are independently selected from hydrogen and substituted or unsubstituted aliphatic.

12. The method of claim 1, wherein the proteasome inhibitor is administered orally.

13. A method of increasing the level of gemini of coiled bodies (gems) of SMN protein in a patient comprising administering a therapeutically effective
amount of at least one proteasome inhibitor or a pharmaceutically acceptable salt, isomer, prodrug, analog, metabolite or derivative thereof.

14. The method of claim 13, wherein said proteasome inhibitor is selected from the group consisting of peptide aldehydes, peptide vinyl sulfones, peptide boronates, peptide epoxiketones, β-lactones or a pharmaceutically acceptable salt, isomer, prodrug, analog, metabolite or derivative thereof.

15. The method of claim 13, wherein said proteasome inhibitor is bortezomib (Velcade®).

16. The method of claim 13, wherein said proteasome inhibitor is lactacystin.

17. The method of claim 13, wherein said proteasome inhibitor is omuralide.

18. The method of claim 13, wherein said proteasome inhibitor is antiprotealide.

19. The method of claim 13, wherein said proteasome inhibitor is epoxomicin.

20. The method of claim 13, wherein said proteasome inhibitor is eponemycin.

21. The method of claim 13, wherein said proteasome inhibitor of the present invention is represented by formula (I): 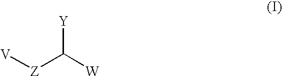
Wherein:
W is: 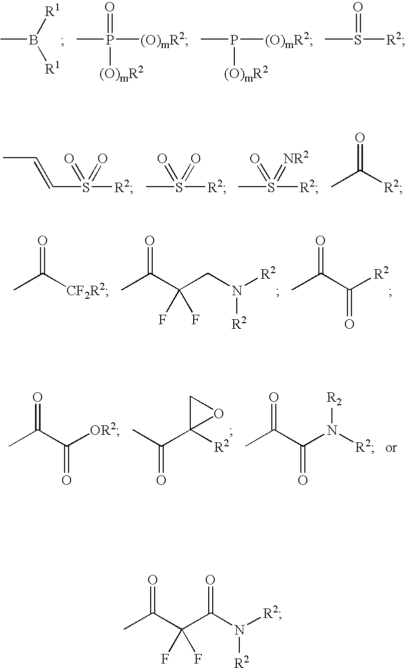
m is 0 or 1;
each R¹ is hydroxy, alkoxy, or aryloxy, or each R¹ is an oxygen atom and together with the boron, to which they are each bound, form a 5-7 membered monocylic, bicyclic, tricyclic or polycyclic ring, wherein the ring is optionally substituted with halogen, N, S, or O;
each R² is independently hydrogen, unsubstituted or substituted, saturated or unsaturated aliphatic, unsubstituted or substituted aryl, unsubstituted or substituted heteroaryl, unsubstituted or substituted cycloalkyl, or unsubstituted or substituted heterocycle; or two R² groups, which are bound to the same nitrogen atom, form together with that nitrogen atom, a 5-7 membered monocyclic heterocyclic ring system optionally substituted with halogen, N, S or O;
Y is a substituted or unsubstituted, saturated or unsaturated aliphatic, unsubstituted or substituted cycloalkyl, unsubstituted or substituted aryl, unsubstituted or substituted heteroaryl;
Z is selected from: 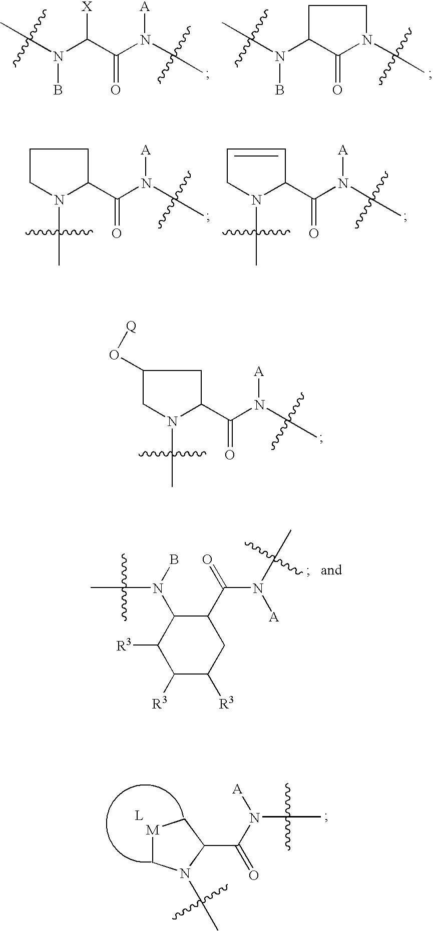
A and B are independently selected from hydrogen, and substituted or unsubstituted aliphatic;
X is a substituted or unsubstituted, saturated or unsaturated aliphatic, unsubstituted or substituted cycloalkyl, unsubstituted or substituted aryl, unsubstituted or substituted heteroaryl;
Q is a substituted or unsubstituted aryl, substituted or unsubstituted heteroaryl;
or a substituted or unsubstituted, saturated or unsaturated aliphatic;
R³ are hydrogen; or two adjacent R³ are bound together to form substituted or unsubstituted aryl and the other R³ is hydrogen;
V is an acyl, a substituted or unsubstituted, saturated or unsaturated aliphatic, unsubstituted or substituted cycloalkyl, unsubstituted or substituted aryl, unsubstituted or substituted heteroaryl;
with the proviso that formula (I) is not

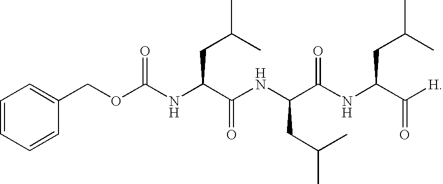

22. The method of claim 13, wherein said proteasome inhibitor of the present invention is represented by formulae (II) and (III): 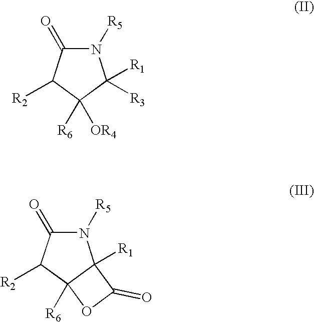
Wherein:
R₁, R₂ and R₆ are independently selected from hydrogen, substituted or unsubstituted, saturated or unsaturated aliphatic;
R₃ is an acyl, a substituted or unsubstituted, saturated or unsaturated aliphatic;
R₄ and R₅ are independently selected from hydrogen and substituted or unsubstituted aliphatic.

23. The method of claim 13 wherein the proteasome inhibitor is administered orally.
